(19) United States
(12) Patent Application Publication
(10) Pub. No.: US 2007/0224216 A1
(43) Pub. Date: Sep. 27, 2007

(54) Methods and Compositions Related to Antiviral Therapy Using Algae and Cyanobacteria

(76) Inventor: Jane Teas, Columbia, SC (US)

Correspondence Address: NEEDLE & ROSENBERG, P.C., SUITE 1000, 999 PEACHTREE STREET, ATLANTA, GA, 30309-3915, US

(21) Appl. No.: 11/579,572

(22) PCT Filed: May 4, 2005
(86) PCT No.: PCT/US05/15698

§ 371 Date: Jan. 11, 2007

Related U.S. Application Data
(60) Provisional application No. 60/567,992, filed on May 4, 2004.

(51) Int. Cl. A61K 36/02; A61K 36/00

(52) U.S. Cl. 424/195.17

(57) ABSTRACT

Disclosed are methods and compositions related to treating and preventing viral infection.

### I. CROSS REFERENCE TO RELATED APPLICATIONS

This application claims priority to U.S. Provisional Application 60/567,992, filed May 4, 2004.

### II. SUMMARY OF THE INVENTION

In accordance with the purposes of this invention, as embodied and broadly described herein, this invention, in one aspect, relates to compositions comprising cyanobacteria and one or more types of algae. Also disclosed are methods for treating or preventing a viral invention in a subject.

Additional advantages of the invention will be set forth in part in the description which follows or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### III. DETAILED DESCRIPTION

The present invention may be understood more readily by reference to the following detailed description of preferred embodiments of the invention and the Examples included therein and their previous description.

Before the present compounds, compositions, articles, devices, and/or methods are disclosed and described, it is to be understood that this invention is not limited to specific synthetic methods, specific compositions, or to particular formulations, as such may, of course, vary. It is also to be understood that the terminology used herein is for the purpose of describing particular embodiments only and is not intended to be limiting.

Throughout this application, various publications are referenced. The disclosures of these publications in their entireties are hereby incorporated by reference into this application in order to more fully describe the state of the art to which this invention pertains. The references disclosed are also individually and specifically incorporated by reference herein for the material contained in them that is discussed in the sentence in which the reference is relied upon. Furthermore, references may be cited along with a letter, such as (3). This letter refers to particular reference list disclosed herein, designated with the letter. Furthermore, should a letter not be associated with a reference number, it will be clear to the skilled artisan, from the context and the potential references, which reference is being relied upon.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Disclosed are the components to be used to prepare the disclosed compositions as well as the compositions themselves and to be used within the methods disclosed herein. These and other materials are disclosed herein, and it is understood that when combinations, subsets, interactions, groups, etc. of these materials are disclosed that while specific reference of each various individual and collective permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus means for example, that combinations A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, combinations of the various algae and cyanobacteria discussed herein, as well as steps in methods of making and using the disclosed compositions. Thus, if there are a variety of additional steps that can be performed it is understood that each of these additional steps can be performed with any specific embodiment or combination of embodiments of the disclosed methods.

### A. GENERAL

Disclosed herein are compositions comprising a combination of cyanobacteria and one or more types of algae, as well as methods of using the same. Algal extracts have been shown to inhibit HIV in cell culture and in animal studies. In populations where brown algae are eaten, most notably in Japan and Korea, the prevalence of HIV/AIDS is about one in a million. HIV/AIDS incidence and prevalence in Eastern Asia (≈1/10,000 adults in Japan and Korea), compared to Africa (≈1/10 adults) show that differences in IV drug use and sexual behavior are insufficient to explain the 1000-fold variation. Even in Africa, AIDS/HIV rates vary. Along the shores of Lake Chad, among the Kanembu tribe, where people eat Spirulina, a blue-green algae (also known as cyanobacteria, referred to throughout alternatively), the incidence of AIDS ranges between 2-4%, and has done so for over 20 years. Average daily algae consumption in Asia and Africa ranges between 1 to 2 tablespoons (3-13 grams). Regular consumption of dietary algae can help prevent viral infection and suppress viral load among those infected.

Evidence from people who have taken seaweed in capsules or Spirulina for the treatment of HIV/AIDS show that symptoms of HIV decrease with algae consumption. Consuming algae before exposure to HIV can increase the viral dose needed for infection, continuing to eat algae can reduce viral replication once infection has taken place, and dietary algae can stimulate the immune system in a broad spectrum manner that could enhance both HIV resistance and complementary immune defenses. Consumption of algae (Spirulina, Undaria, or Sargassum) is associated with decreased rates of HIV infection.

Daily consumption of certain algae and cyanobacteria in combination can decrease the number of HIV copies/mL and increase the number of CD4 cells, among other positive effects. In addition, the combination can work synergistically to enhance immune function and increase inhibition of HIV replication. These methods of treatment can prolong the time until Highly Active Antiretroviral Therapy (HAART) is necessary, or prevent it altogether. Algae are nontoxic, and widely available. Both algae and cyanobacteria are inexpensive, and both can be grown easily.

### B. COMPOSITIONS

Disclosed are compositions comprising cyanobacteria and one or more types of algae. Both algae and cyanobacteria (such as Spirulina) are nontoxic and safe. Spirulina has a long history of use by humans, both as food as a dietary supplement. Toxicity studies have shown it to be safe and to meet or exceed all national foods standards (Belay, 2002). The Food and Drug Administration classify brown seaweeds as “Generally Regarded As Safe” (GRAS). It is eaten daily by millions of people around the world.

A related mechanism of action for both seaweed and Spirulina is its antibiotic activity. Supplementation with algae protects HIV-infected individuals from a variety of opportunistic bacterial infections (Belay, 2002; Vlachos, 1996).

Although brown algae and Spirulina appear dissimilar in terms of color (brown vs. blue-green) and habitat (ocean vs. alkaline lakes), they have two characteristics in common: both have sulfated polysaccharide cell wall constituents and both utilize negative ion pumps to maintain homeostasis in high pH environments. Furthermore, seaweed extracts do not stimulate natural killer cell activity but do stimulate CD4 proliferation, whereas Spirulina stimulates NK cell activity but does not stimulate CD4 proliferation. Both inhibit HIV-CD4 fusion. The two algae, when given at the same time, can enhance immune function, for example, by inhibiting HIV-CD4 binding.

1. Cyanobacteria

The term “cyanobacteria,” as used herein, refers to prokaryotic organisms formerly classified as the blue-green algae. Cyanobacteria are a large and diverse group of photosynthetic bacteria which comprise the largest subgroup of Gram-negative bacteria. Cyanobacteria were classified as algae for many years due to their ability to perform oxygen-evolving photosynthesis. (Curtis, “Cyanobacteria, Molecular Genetics”, Encyclopedia of Microbiology, vol. 1, 627 (1992)). While many cyanobacteria have a mucilaginous sheath which exhibits a characteristic blue-green color, the sheaths in different species may also exhibit colors including light gold, yellow, brown, red, emerald green, blue, violet, and blue-black. (Raven et al., Biology of Plants, Fourth Edition, 183-185, (1986)), included herein by reference. Cyanobacteria include Microcystis aeruginosa, Trichodesmium erythraeum, Aphanizomenon flos-aquae, Spirulina, and Anabaena flos-aquae. One of ordinary skill in the art can identify other cyanobacteria that are safe for consumption and can be used with the compositions and methods disclosed herein.

The cyanobacterium Spirulina has long been valued as a food source; it is high in protein, and can be cultivated in easily. In tropical countries, it is a very important part of the diet, and was eaten regularly by the Aztecs; it is also served in several Oriental dishes. In the US, the popularity of Spirulina is primarily as a “health food,” being sold in stores as a dried powder or in tablet form.

An in vitro study of human peripheral blood mononuclear cells reported that aqueous Spirulina extract (calcium spirulan) at 40 μg/mL almost completely inhibited HIV-1 adsorption and penetration (Ayehunie, 1998). No toxicity to uninfected cells was noted. Patients with AIDS who have used Spirulina have reported an increased sense of well-being. Furthermore, a clinical study of 50 ml oral Spirulina given to 12 healthy men for three months resulted in enhanced immune response, including increased interferon production and natural killer cell cytotoxicity (Hirahashi, 2002).

Furthermore, two weeks of oral Spirulina (50 ml/d of a Spirulina drink containing 40% Spirulina hot water extract) given to healthy 40 year-old male volunteers resulted in increased IFN-γ and natural killer cell activity (Belay, 2002 Hirahashi, 2002).

2. Algae

Algae represent a large, heterogeneous group of primitive photosynthetic organisms which occur throughout all types of aquatic habitats and moist terrestrial environments. (Nadakavukaren et al., Botany, An Introduction to Plant Biology, 324-325, (1985)). The term “algae”, as used herein, refers to the following algal divisions: Chlorophyta (green algae), Euglenophyta (euglenoids), Chrysophyta (golden and yellow-green algae, diatoms), Phaeophyta (brown algae), Pyrrophyta (dinoflagellates), and Rhodophyta (red algae). Such divisions are described more fully in Nadakavukaren et al., Botany, An Introduction to Plant Biology, 324-349, (1985), Brock et al., Biology of Microorganisms, 815-817, (1991), and Bold et al., Introduction to the Algae, 1-32, (1978), which are incorporated herein by reference.

Green algae include Chlorella and Chlorococcum. Euglenoids include Euglena mesnili, Trachelomonas armata, and Phacus pleuronectes. Golden algae include Dinobryon, spp. and Synura, spp. Diatoms include Nitzschia pungens, f. maltiseries, and Nitzschia pseudodelicatissima. Brown algae include Pilayella littoralis (zoospores). Dinoflagellates include Dinophysis acuminata, Dinophysis norvegica, Gymnodinium, and Gonyaulax catenella. Red algae include Rhodymenia, spp. and Bangia, spp. Preferred algae are Chlorophyta such as Chlorella and Chlorococcum; Chrysophyta such as Dinobryon and Synura; and combinations thereof.

“Algae,” “kelp,” and “seaweed” are used interchangeably throughout. The kelps generally include the many large brown seaweeds and are among the most familiar forms found on North American coasts. Some have fronds up to 200 ft (61 m) long, e.g., the Pacific coast Nereocystis and Macrocystis, found also off the Cape of Good Hope. Common Atlantic species include Laminaria and Agarum (devil's apron). The kelps are a source of salts of iodine and potassium and, to a lesser extent, other minerals. When the seaweed is burned, the soluble mineral compounds are removed from the ashes (also called kelp) by washing. They are used chiefly as chemical reagents and for dietary deficiencies in people and in livestock. Kelp is also a commercial source of potash, fertilizer, and medicines made from its vitamin and mineral content. Kelps are especially abundant in Japan, and various foods known as kombu are made from them.

The brown algae of the genus Sargassum is also called gulfweed. They inhabit warm ocean regions and are commonly found floating in large patches in the Sargasso Sea and in the Gulf Stream. Although it was formerly thought to cover the whole Sargasso Sea, making navigation impossible, it has since been found to occur only in drifts. Numerous berrylike air sacs keep the branching plant afloat. The thick masses of gulfweed provide the environment for a distinctive and specialized group of marine forms, many of which are not found elsewhere. Other brown algae includes Undaria and Alaria.

The safety of brown seaweeds depends on their iodine content. The popular Undaria (“wakame”), Alaria (American “wakame”) and the less commonly eaten Sargassum, have safe levels of iodine (40-100 ug/g). This is not true of Laminaria (“kombu”) Hizikia, or Eisenia (“arame”), which all contain high iodine levels which can cause iodine sensitive individuals to develop transient thyrotoxicosis. The maximum tolerated dose of iodine is 1,000 μg/day, and the background level of iodine intake is about 250 μg/day. Five grams of Undaria provides an additional 200 μg/day.

Algae, unlike narrowly targeted drugs, have been shown to exert a variety of health effects, including antiviral, antibacterial, antioxidant, anti-inflammatory, immune enhancing, probiotic, and cholesterol-lowering effects. As whole foods, rather than isolated fractions, the full spectrum of possible biochemical pathways for modulating health in diverse ways, are available to reduce HIV infection. In addition to direct effects on viruses in culture, dietary algal extracts have shown a broad spectrum of immune enhancement in vivo and in vitro. These include increased production of interleukin-12 and interferon-1β in the presence of viral infection (Hirahashi, 2002), stimulate natural killer cell stimulation (Hirahashi, 2002), and B cell stimulation (Shan, 1999).

Given HIV infection, consumption of algae is associated with reduced viral replication and improvements in CD4 counts, and decreased HIV-related symptoms and opportunistic infections. Algae consumption can also prevent or slow progression of HIV-infection to AIDS. Patients with AIDS to whom seaweed has been given as a food have report diminished AIDS-related symptoms of diarrhea, respiratory distress, anorexia, fatigue, and insomnia. Furthermore, the algal compositions disclosed herein can be combined with conventional therapy to achieve maximum results.

In vitro studies of seaweed report that concentrations of 50-1,000 μg/ml in HIV infected MT-4 cells, resulted in the disappearance of almost all HIV infected cells (Muto, 1992). The same dose strongly inhibited HIV reverse transcriptase activity. Viva Natural, a commercial water extract of Undaria, suppressed replication of Rauscher virus in BALB/3T3 cells and inhibited syncytia formation and when used in vivo, was as effective as AZT in treating the Rauscher murine retrovirus in mice (Furusawa, 1991). A hot water extract of Sargassum, another brown seaweed, was effective in inhibiting HIV infection in vitro (Hoshino, 1998). Various seaweed extracts have also shown anti-HIV activity (Witvrouw, 1997).

In a human trial using the seaweed Undaria, 15 HSV infected patients were reported to have faster healing and decreased reactivation of HSV (Cooper, 2002).

In another study, four healthy men took 2 g/d of Undaria for 14 days, and comparison of pre and post Undaria ingestion revealed a 12% increase in CD4 cell counts. Normal range was 400-1100 cells/ml. The initial average CD4 counts were 845 (range 657 to 1089/ml). Post Undaria CD4 counts were 944 cells/ml (range 792 to 1232).

Dietary algal extracts have a broad spectrum of immune enhancement in vivo. In a randomized double-blinded study conducted in 30 healthy postmenopausal women who took seaweed (Alaria, a closely related seaweed to Undaria) for 6 weeks, two women with long standing psoriasis experienced relief of symptoms during the seaweed supplementation and nine reported having more energy when they were taking seaweed.

In a second preliminary study of the acute effects of 5 g of Undaria in 10 healthy volunteers, P-selectin, a marker of cell-cell adhesion, decreased significantly. This appears to be relevant to the decrease in CD4-HIV fusion reported in vitro

Furthermore, as discussed above, seaweed contains iodine, and iodine has HIV-antiviral activity topically (Kawana, 1997), and it has been shown that ingested iodine can be active internally. Polysaccharide-bound iodine provides iodine slowly and in a non-irritating form. In a study of 111 patients, iodine/polysaccharide/lithium monthly injections were used as a therapy for AIDS (Armenicum, 2001). Eighty of the patients provided complete data and continued the treatment for 12 weeks. A 40% increase in CD4 cells and a 1.2 log decrease in viral load by 12 weeks was observed, with the greatest change in the first four weeks.

Another pathway involves algal antioxidant properties that include high levels of carotene and phycocyanin, an antioxidant pigment protein that characterizes blue-green algae in Spirulina (Pinero, 2001; Fike, 2001). A carotenoid almost unique to brown seaweeds, fucoxanthin can be used as a marker for Undaria ingestion. Phycocyanin (for Spirulina) and fucoxanthin (for Undaria) can be detected using fluorometric analysis of spot urine (Beutler, 2002).

Seaweed derived fucans have been shown to modulate interleukin-1α, tumor necrosis factor α, interleukin-6, and interleukin-8, as well as cytokine production by lipopolysaccharide-stimulated monocytes and inhibited monocyte-LPS membrane binding (Anastase-Ravion, 2002). In a separate study, seaweed extracts were shown to stimulate the proliferation of human lymphocytes in vitro. Cytotoxic T lymphocytes were stimulated, as was the production of immunoglobulin production by B cells and tumor necrosis factor by monocytes (Shan, 1999). An earlier study by Okai showed increased ingestive activity of phagocytic cells against S. aureus. A kelp extract (Laminaria japonica) increased antibody production of B lymphocytes of C3H/HeJ mice (Okai, 1996). Both IL-1α and TNFα production by seaweed treated phagocytic cells was increased about fourfold at the lowest dose of seaweed extract (mg^{ml-1}), and there was a dose response increase with increasing seaweed concentration. In a study of cows fed endophyte infected grass, there was a significantly decreased monocyte phagocytic activity and major histocompatibility complex class II expression, but that when the grass was treated with a seaweed extract, these effects were reversed (p<0.05) (Saker, 2001).

Studies done in vitro show that the negatively charged (polyanionic) crude algal extracts can be used in anti-HIV therapies (Luscher-Mattli, 2000; Schaeffer, 2000; Witrouw, 1997). Specifically, algal polyanions bind competitively with the positively charged sites on the V3 loop of the CD4 cell surface, resulting in specific disruption in enveloped viruses such as HIV in virus-CD4 fusion (Witrouw, 1997). Studies of Spirulina and seaweed extracts report HIV inhibition with no toxicity to uninfected cells (Ayehunie, 1998, Muto, 1992 Hoshino, 1998; Witvrouw, 1997). Various crude seaweed extracts have also been shown to improve immune function in vitro (Anastase-Ravion, 2002; Shan, 1999; Okai, 1996) and in vivo (Saker, 2001). Clinical data are available for whole seaweed (Undaria) in 15 Herpes simplex virus-infected patients (Cooper, 2002). Patients reported decreased healing time and reduced HSV reactivation. Two weeks of oral Spirulina supplementation in healthy volunteers resulted in increased IFN-γ and natural killer (NK) cell activity (Belay, 2002; Hirahashi, 2002.)

Algae in the gut can provide direct inhibition of viral entry to gut mucosal surfaces, whilst uptake of antiviral components can provide direct inhibition of HIV-T cell interactions in blood. HIV-infected persons can derive more direct antiviral activity from ingesting algae due to a greater uptake of material through more porous gut. Gut-associated lymph tissue or GALT, take up large molecules, which are then presented to resident specialized immune cells and can be transported in the lymph (Weiner, 1988). For example, T cells in gut cryptopatches are important in defense against HSV in mice whereas Peyer's patch interactions have an important role in both T cell maturation and B cell presentation (Sciammas, 1969). The presence of algal components can stimulate proliferation or activation of immune cells in these areas.

3. Extracts, Derivatives, Lysates, and Fractions

Disclosed herein are compositions comprising fractions of cyanobacteria and one or more types of algae. Also disclosed are extracts, lysates, or derivatives of cyanobacteria and one or more types of algae. The extracts, lysates, and derivatives can be active or inactive. In one example, the extract of the algae comprises algal polyanions.

The principal overall objective disclosed herein is to provide anti-viral compositions, peptides and derivatives thereof, and broad medical uses thereof, including prophylactic and/or therapeutic applications against viruses. Antiviral activity has been observed in certain extracts from cultured cyanobacteria tested in an anti-HIV screen.

Cyanobacteria and other types of algae were specifically chosen for anti-HIV screening because they had been known to produce a wide variety of structurally unique and biologically active non-nitrogenous and amino acid-derived natural products (Faulkner, Nat. Prod. Rep. 11, 355-394, 1994; and Glombitza et al., in Algal and Cyanobacterial Biotechnology, Cresswell, R. C., et al. eds., 1989, pp. 211-218). Cyanobacteria, photosynthetic procaryotic organisms, are significant producers of cyclic and linear peptides (molecular weight generally <3 kDa), which often exhibit hepatotoxic or antimicrobial properties (Okino et al., Tetrahedron Lett. 34, 501-504, 1993; Krishnamurthy et al., PNAS USA 86, 770-774, 1989; Sivonen et al., Chem. Res. Toxicol. 5, 464-469, 1992; Carter et al., J. Org. Chem. 49, 236-241, 1984; and Frankmolle et al., J. Antibiot. 45, 1451-1457, 1992). Sequencing studies of higher molecular weight cyanobacterial peptides and proteins have generally focused on those associated with primary metabolic processes or ones that can serve as phylogenetic markers (Suter et al., FEBS Lett. 217, 279-282, 1987; Rumbeli et al., FEBS Lett. 221, 1-2, 1987; Swanson et al., J. Biol. Chem. 267, 16146-16154, 1992; Michalowski et al., Nucleic Acids Res. 18, 2186, 1990; Sherman et al., in The Cyanobacteria, Fay et al., eds., Elsevier: New York, 1987, pp. 1-33; and Rogers, in The Cyanobacteria, Fay et al., eds., Elsevier: New York, 1987, pp. 35-67). In general, proteins with antiviral properties had not been associated with cyanobacterial sources.

In the bioassay-guided strategy, initial selection of the extract for fractionation, as well as the decisions concerning the overall chemical isolation method to be applied, and the nature of the individual steps therein, is determined by interpretation of biological testing data. The anti-HIV screening assay (e.g., see Boyd, 1988, supra; Weislow et al., J. Natl. Cancer Inst. 81, 577-586, 1989), which is used to guide the isolation and purification process, measures the degree of protection of human T-lymphoblastoid cells from the cytopathic effects of HIV. Fractions of the extract of interest are prepared using a variety of chemical means and are tested blindly in the primary screen. Active fractions are separated further, and the resulting subfractions are likewise tested blindly in the screen. This process is repeated as many times as necessary in order to obtain the active compound(s), i.e., antiviral fraction(s) representing pure compound(s), which then can be subjected to detailed chemical analysis and structural elucidation.

Cyanovirin is an example of a cyanobacterial extract that can be used with the methods and compositions disclosed herein (see U.S. Pat. No. 6,780,847). It is used generically to refer to a native cyanovirin or any related, functionally equivalent (i.e., antiviral) protein, peptide or derivative thereof. By definition, in this context, a related, functionally equivalent protein, peptide or derivative thereof a) contains a sequence of at least nine amino acids directly homologous with any sub-sequence of nine contiguous amino acids contained within a native cyanovirin, and, b) is capable of specifically binding to a virus, in particular an influenza virus or a retrovirus, more specifically a primate immunodeficiency virus, more specifically HIV-1, HIV-2 or SIV, or to an infected host cell expressing one or more viral antigen(s), more specifically an envelope glycoprotein, such as gp120, of the respective virus.

Preferably, the composition, fraction, lysate, or derivative thereof comprises an amino acid sequence that is substantially homologous to that of an antiviral protein. By “substantially homologous” is meant sufficient homology to render the protein, peptide or derivative thereof antiviral, with antiviral activity characteristic of an antiviral protein. At least about 50% homology, preferably at least about 75% homology, and most preferably at least about 90% homology should exist. “Immunological reagent” will be used to refer to an antibody, an immunoglobulin, and an immunological recognition element. An immunological recognition element is an element, such as a peptide, which facilitates, through immunological recognition, isolation and/or purification and/or analysis of the protein or peptide to which it is attached. A fusion protein is a type of conjugate, wherein a protein is coupled to another protein(s) having any desired properties or effector functions, such as cytotoxic or immunological properties, or other desired properties, such as to facilitate isolation, purification or analysis of the fusion protein.

4. Supplements

Also disclosed herein are nutritional (also referred to as dietary throughout the application) supplements. A nutritional supplement is any compound or composition that can be administered to or taken by a subject to provide, supply, or increase an effect, such as an antiviral property. In one aspect, disclosed herein are nutritional supplements comprising any of the compositions disclosed herein. For example, a nutritional supplement can comprise a cyanobacteria and one or more types of algae, or fractions, extracts, lysates, or derivatives thereof. The nutritional supplement can comprise any amount of the compositions disclosed herein, but will typically contain an amount determined to supply a subject with a desired dose of the composition. The exact amount of composition required in the nutritional supplement will vary from subject to subject, depending on the species, age, weight and general condition of the subject, the severity of the dietary deficiency being treated, the particular mode of administration, and the like. Thus, it is not possible to specify an exact amount for every nutritional supplement. However, an appropriate amount can be determined by one of ordinary skill in the art using only routine experimentation given the teachings herein.

In one specific example, a nutritional supplement can comprise from about 1 to about 20 grams of algae, and 1 to about 20 grams of cyanobacteria, or fractions, extracts, lysates, or derivatives thereof. Also disclosed are amounts ranging from about 20 to about 1500 grams, from about 50 to about 200 grams. In another example, the nutritional supplement can comprise from about 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 325, 350, 375, 400, 500, 600, 700, 800, 900, 1000, 1500, 2000, 2500, or 3000 grams of algae, and about 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 325, 350, 375, 400, 500, 600, 700, 800, 900, 1000, 1500, 2000, 2500, or 3000 grams of cyanobacteria or fractions, extracts, lysates, or derivatives thereof, where any of the stated values can form an upper or lower endpoint when appropriate. Furthermore, the algae and cyanobacteria or fractions, extracts, lysates, or derivatives thereof can be given in the same supplement, or simultaneously in different supplements, or in adjacent supplements taken near the same time, such as within about 10, 20, 30, 40, or 50 seconds, or within about 1, 2, 3, 4, 5, 6, 7, 8, 9, 10, 11, 12, 13, 14, 15, or 30 minutes, or within about 1, 2, 3, 4, 5, 6, 7, 8, 9, 10, 11 or 12 hours, or within 24 hours. Also, different types of algae can be administered simultaneously. Any types of algae known to those of skill in the art can be administered according to the methods disclosed herein.

The nutritional supplement can also comprise other nutrient(s) such as vitamins other trace elements, minerals, and the like. Further, the nutritional supplement can comprise other components such as preservatives, antimicrobials, anti-oxidants, chelating agents, thickeners, flavorings, diluents, emulsifiers, dispersing aids, or binders.

The nutritional supplements are generally taken orally and can be in any form suitable for oral administration. For example, a nutritional supplement can typically be in a tablet, gel-cap, capsule, liquid, sachets, or syrup form.

(1) Pharmaceutical Formulation

Also, disclosed herein are pharmaceutical formulations. In one aspect, a pharmaceutical formulation can comprise any of the compositions disclosed herein with a pharmaceutically acceptable carrier. For example, a pharmaceutical formulation can comprise an algae/cyanobacteria composition (or fractions, extracts, lysates, or derivatives thereof) comprising one or more types of algae and one or more types of cyanobacteria, and a pharmaceutically acceptable carrier. The disclosed pharmaceutical formulations can be used therapeutically or prophylactically.

By “pharmaceutically acceptable” is meant a material that is not biologically or otherwise undesirable, i.e., the material may be administered to a subject without causing any undesirable biological effects or interacting in a deleterious manner with any of the other components of the pharmaceutical formulation in which it is contained. The carrier would naturally be selected to minimize any degradation of the active ingredient and to minimize any adverse side effects in the subject, as would be well known to one of skill in the art.

Pharmaceutical carriers are known to those skilled in the art. These most typically would be standard carriers for administration of drugs to humans, including solutions such as sterile water, saline, and buffered solutions at physiological pH. Suitable carriers and their formulations are described in Remington: The Science and Practice of Pharmacy (19th ed.) Gennaro, ed., Mack Publishing Company, Easton, Pa., 1995, which is incorporated by reference herein for its teachings of carriers and pharmaceutical formulations. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the disclosed compounds, which matrices are in the form of shaped articles, e.g., films, liposomes, microparticles, or microcapsules. It will be apparent to those persons skilled in the art that certain carriers can be more preferable depending upon, for instance, the route of administration and concentration of composition being administered. Other compounds can be administered according to standard procedures used by those skilled in the art.

Pharmaceutical formulations can include additional carriers, as well as thickeners, diluents, buffers, preservatives, surface active agents and the like in addition to the compounds disclosed herein. Pharmaceutical formulations can also include one or more additional active ingredients such as antimicrobial agents, antiinflammatory agents, anesthetics, and the like.

The pharmaceutical formulation can be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed compounds can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, fish oils, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Pharmaceutical formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Pharmaceutical formulations for oral administration include, but are not limited to, powders or granules, suspensions or solutions in water or non-aqueous media, capsules, sachets, or tablets. Thickeners, flavorings, diluents, emulsifiers, dispersing aids or binders may be desirable.

Some of the formulations can potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

(2) Delivery Devices

Any of the compositions described herein can be incorporated into a delivery device. Examples of delivery devices include, but are not limited to, microcapsules, microspheres, nanospheres or nanoparticles, liposomes, noisome, nanoerythrosome, solid-liquid nanoparticles, gels, gel capsules, tablets, lotions, creams, sprays, emulsions, or powders. Other examples of delivery devices that are suitable for non-oral administration include pulmospheres. Examples of particular delivery devices useful herein are described below.

The disclosed compounds can be incorporated into liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The disclosed compositions in liposome form can contain, in addition to a compound disclosed herein, stabilizers, preservatives, excipients, and the like. Examples of suitable lipids are the phospholipids and the phosphatidyl cholines (lecithins), both natural and synthetic. Methods of forming liposomes are known in the art. See, e.g., Prescott, Ed., Methods in Cell Biology, Volume XIV, Academic Press, New York, p. 33 et seq., 1976, which is hereby incorporated by reference herein for its teachings of liposomes and their preparation.

In other examples, the liposomes can be cationic liposomes (e.g., DOTMA, DOPE, DC cholesterol) or anionic liposomes. Liposomes can further comprise proteins to facilitate targeting a particular cell, if desired. Administration of a composition and a cationic liposome can be administered to the blood afferent to a target organ or inhaled into the respiratory tract to target cells of the respiratory tract. Regarding liposomes, see, e.g., Brigham, et al., Am J Resp Cell Mol Biol 1:95-100, 1989; Felgner, et al., Proc Natl Acad Sci USA 84:7413-7, 1987; and U.S. Pat. No. 4,897,355, which are incorporated by reference herein for their teachings of liposomes. As one example, delivery can be via a liposome using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. Liposomes where the diffusion of the compound or delivery of the compound from the liposome is designed for a specific rate or dosage can also be used.

As described herein, niosomes are delivery devices that can be used to deliver the compositions disclosed herein. Noisomes are multilamellar or unilamellar vesicles involving non-ionic surfactants. An aqueous solution of solute is enclosed by a bilayer resulting from the organization of surfactant macromolecules. Similar to liposomes, noisomes are used in targeted delivery of, for example, anticancer drugs, including methotrexate, doxorubicin, and immunoadjuvants. They are generally understood to be different from transferosomes, vesicles prepared from amphiphilic carbohydrate and amino group containing polymers, e.g., chitosan.

As described herein, nanoerythrosomes are delivery devices that can be used to deliver the compositions disclosed herein. Nanoerythrosomes are nano-vesicles made of red blood cells via dialysis through filters of defined pore size. These vesicles can be loaded with a diverse array of biologically active molecules, including proteins and the compositions disclosed herein. They generally serve as ideal carriers for antineoplastic agents like bleomycin, actinomycin D, but can be used for steroids, other lipids, etc.

Artificial red blood cells, as described herein, are further delivery devices that can be used to deliver the compositions disclosed herein. Artificial red blood cells can be generated by interfacial polymerization and complex emulsion methods. Generally, the “cell” wall is made of polyphtaloyl L-lysine polymer/polystyrene and the core is made of a hemoglobin solution from sheep hemolysate. Hemoglobin loaded microspheres typically have particle sizes of from about 1 to about 10 mm. Their size, flexibility, and oxygen carrying capacity is similar to red blood cells.

Solid-lipid nanoparticles, as described herein, are other delivery devices that can be used to deliver the compositions disclosed herein. Solid-lipid nanoparticles are nanoparticles, which are dispersed in an aqueous surfactant solution. They are comprised of a solid hydrophobic core having a monolayer of a phospholipid coating and are usually prepared by high-pressure homogenization techniques. Immunomodulating complexes (ISCOMS) are examples of solid-lipid nanoparticles. They are cage-like 40 nm supramolecular assemblies comprising of phospholipid, cholesterol, and hydrophobic antigens and are used mostly as immunoadjuvants. For instance, ISCOMs are used to prolong blood-plasma levels of subcutaneously injected cyclosporine.

Microspheres and micro-capsules, as described herein, are yet other delivery devices that can be used to deliver the compositions disclosed herein. In contrast to liposomal delivery systems, microspheres and micro-capsules typically do not have an aqueous core but a solid polymer matrix or membrane. These delivery devices are obtained by controlled precipitation of polymers, chemical cross-linking of soluble polymers, and interfacial polymerization of two monomers or high-pressure homogenization techniques. The encapsulated compound is gradually released from the depot by erosion or diffusion from the particles. Successful formulations of short acting peptides, such as LHRH agonists like leuprorelin and triptoreline, have been developed. Poly(lactide co-glycolide (PLGA) microspheres are currently used as monthly and three monthly dosage forms in the treatment of advanced prostrate cancer, endometriosis, and other hormone responsive conditions. Leuprolide, an LHRH superagonist, was incorporated into a variety of PLGA matrices using a solvent extraction/evaporation method. As noted, all of these delivery devices can be used in the methods disclosed herein.

Pulmospheres are still other examples of delivery devices that can be used herein. Pulmospheres are hollow porous particles with a low density (less than about 0.1 gm/mL). Pulmospheres typically have excellent re-dispersibility and are usually prepared by supercritical fluid condensation technology. Co-spray-drying with certain matrices, such as carbohydrates, human serum albumin, etc., can improve the stability of proteins and peptides (e.g., insulin) and other biomolecules for pulmonary delivery. This type of delivery could be also accomplished with micro-emulsions and lipid emulsions, which are ultra fine, thin, transparent oil-in-water (o/w) emulsions formed spontaneously with no significant input of mechanical energy. In this technique, an emulsion can be prepared at a temperature, which must be higher than the phase inversion temperature of the system. At elevated temperature the emulsion is of water-in-oil (w/o) type and as it cools at the phase inversion temperature, this emulsion is inverted to become o/w. Due to their very small inner phase, they are extremely stable and used for sustained release of steroids and vaccines. Lipid emulsions comprise a neutral lipid core (i.e., triglycerides) stabilized by a monolayer of amphiphilic lipid (i.e., phospholipid) using surfactants like egg lecithin triglycerides and miglyol. They are suitable for passive and active targeting.

There are other oral delivery systems under investigation that are based on osmotic pressure modulation, pH modulation, swelling modulation, altered density and floating systems, mucoadhesiveness etc. These formulations and time-delayed formulations to deliver drugs in accordance with circadian rhythm of disease that are currently in use or investigation can be applied for delivery of the compositions disclosed herein.

(a) Microcapsules

In one aspect disclosed herein, the disclosed compositions can be incorporated into microcapsules. In one aspect, the microcapsule comprises an agglomeration of primary microcapsules and the composition described herein, each individual primary microcapsule having a primary shell, wherein the composition is encapsulated by the primary shell, wherein the agglomeration is encapsulated by an outer shell. These microcapsules are referred to herein as “multicore microcapsules.”

In another aspect, described herein are microcapsules comprising the disclosed compositions, a primary shell, and a secondary shell, wherein the primary shell encapsulates the composition, and the secondary shell encapsulates the loading substance and primary shell. These microcapsules are referred to herein as “single-core” microcapsules.

Optionally, other loading substances can be encapsulated with the composition. The loading substance can be any substance that is not entirely soluble in the aqueous mixture. In one aspect, the loading substance is a solid, a hydrophobic liquid, or a mixture of a solid and a hydrophobic liquid. In another aspect, the loading substance comprises a grease, an oil, a lipid, a drug (e.g., small molecule), a biologically active substance, a nutritional supplement (e.g., vitamins), a flavor compound, or a mixture thereof. Examples of oils include, but are not limited to, animal oils (e.g., fish oil, marine mammal oil, etc.), vegetable oils (e.g., canola or rapeseed), mineral oils, derivatives thereof or mixtures thereof. The loading substance can be a purified or partially purified oily substance such as a fatty acid, a triglyceride or ester thereof, or a mixture thereof. In another aspect, the loading substance can be a carotenoid (e.g., lycopene), a satiety agent, a flavor compound, a drug (e.g., a water insoluble drug), a particulate, an agricultural chemical (e.g., herbicides, insecticides, fertilizers), or an aquaculture ingredient (e.g., feed, pigment).

In one aspect, the loading substance can be an omega-3 fatty acid. Examples of omega-3 fatty acids include, but are not limited to, -linolenic acid (18:3ÿ), octadecatetraenoic acid (18:4ÿ), eicosapentaenoic acid (20:5ÿ) (EPA), docosahexaenoic acid (22:6ÿ) (DHA), docosapentaenoic acid (22:5ÿ) (DPA), eicosatetraenoic acid (20:4ÿ), uncosapentaenoic acid (21:5ÿ), docosapentaenoic acid (22:5ÿ) and derivatives thereof and mixtures thereof. Many types of derivatives of omega-3 fatty acids are well known in the art. Examples of suitable derivatives include, but are not limited to, esters, such as phytosterol esters, branched or unbranched C1-C30 alkyl esters, branched or unbranched C2-C30 alkenyl esters, or branched or unbranched C3-C30 cycloalkyl esters such as phytosterol esters and C1-C6 alkyl esters. Sources of oils can be derived from aquatic organisms (e.g., anchovies, capelin, Atlantic cod, Atlantic herring, Atlantic mackerel, Atlantic menhaden, salmonids, sardines, shark, tuna, etc) and plants (e.g., flax, vegetables, etc) and microorganisms (e.g., fungi and algae).

In one aspect, the loading substance can contain an antioxidant. Examples of antioxidants include, but are not limited to, vitamin E, CoQ10, tocopherols, lipid soluble derivatives of more polar antioxidants such as ascorbyl fatty acid esters (e.g., ascorbyl palmitate), plant extracts (e.g., rosemary, sage and oregano oils), algal extracts, and synthetic antioxidants (e.g., BHT, TBHQ, ethoxyquin, alkyl gallates, hydroquinones, tocotrienols).

A number of different polymers can be used to produce the shell layers of the single and multicore microcapsules. Examples of such polymers include, but are not limited to, a protein, a polyphosphate, a polysaccharide, or a mixture thereof. In another aspect, the shell material used to prepare the single- and multicore microcapsules further comprises In another aspect, the shell material used to prepare the single- and multicore microcapsules further comprises gelatin type A, gelatin type B, polyphosphate, gum arabic, alginate, chitosan, carrageenan, pectin, starch, modified starch, alfa-lactalbumin, beta-lactoglobumin, ovalbumin, polysorbiton, maltodextrins, cyclodextrins, cellulose, methyl cellulose, ethyl cellulose, hydropropylmethylcellulose, carboxymethylcellulose, milk protein, whey protein, soy protein, canola protein, albumin, chitin, polylactides, poly-lactide-co-glycolides, derivatized chitin, chitosan, poly-lysine, various inorganic-organic composites, or any mixture thereof. It is also contemplated that derivatives of these polymers can be used as well. In another aspect, the polymer can be kosher gelatin, non-kosher gelatin, Halal gelatin, or non-Halal gelatin.

In one aspect, the material used to make the shells of the single- or multicore microcapsules is a two-component system made from a mixture of two different types of polymers. In one aspect, the material is a complex coacervate between the polymer components. Complex coacervation is caused by the interaction between two oppositely charged polymers. Processing aids can be included in the shell material (e.g., primary or outer shells). Processing aids can be used for a variety of reasons. For example, they may be used to promote agglomeration of the primary microcapsules, stabilize the emulsion system, improve the properties of the outer shells, control microcapsule size and/or to act as an antioxidant. In one aspect, the processing aid can be an emulsifier, a fatty acid, a lipid, a wax, a microbial cell (e.g., yeast cell lines), a clay, or an inorganic compound (e.g., calcium carbonate). Not wishing to be bound by theory, these processing aids can improve the barrier properties of the microcapsules.

In one aspect, one or more antioxidants can be added to the shell material. Antioxidant properties are useful both during the process (e.g. during coacervation and/or spray drying) and in the microcapsules after they are formed (i.e. to extend shelf-life, etc). Preferably a small number of processing aids that perform a large number of functions can be used. In one aspect, the antioxidant can be a phenolic compound, a plant extract, or a sulphur-containing amino acid. In one aspect, ascorbic acid (or a salt thereof such as sodium or potassium ascorbate) can be used to promote agglomeration of the primary microcapsules, to control microcapsule size and to act as an antioxidant. The antioxidant can be used in an amount of about 100 ppm to about 12,000 ppm, or from about 1,000 ppm to about 5,000 ppm. Other processing aids such as, for example, metal chelators, can be used as well. For example, ethylene diamine tetraacetic acid can be used to bind metal ions, which can reduce the catalytic oxidation of the loading substance.

In one aspect, the primary microcapsules (primary shells) have an average diameter of about 40 nm to about 10 μm, 0.1 μm to about 10 μm, 1 μm to about 10 μm, 1 μm to about 8 μm, 1 μm to about 6 μm, 1 μm to about 4 μm, or 1 μm to about 2 μm, or 1 μm. In another aspect, the multicore microcapsules can have an average diameter of from about 1 μm to about 2000 μm, 20 g/m to about 1000 μm, from about 20 μm to about 100 μm, or from about 30 μm to about 80 μm. In another aspect, the single-core microcapsules have an outer diameter of from 1 μm to 2,000 μm.

The microcapsules described herein generally have a combination of high payload and structural strength. For example, payloads of loading substance can be from 20% to 90%, 50% to 70% by weight, or 60% by weight of the single or multicore microcapsules.

In one aspect, the methods disclosed in U.S. Patent Application Publication No. 2003/0193102, which is incorporated by reference in its entirety, can be used to encapsulate the compositions described herein. It is also contemplated that one or more additional shell layers can be placed on the outer shell of the single or multicore microcapsules. In one aspect, the techniques described in International Publication No. WO 2004/041251 A1, which is incorporated by reference in its entirety, can be used to add additional shell layers to the single and multicore microcapsules.

(3) Targeted Delivery

The compositions disclosed herein can be targeted to a particular cell type, such as islets cells, via antibodies, receptors, or receptor ligands. The following references are examples of the use of this technology to target specific tissue (Senter, et al., Bioconjugate Chem 2:447-51, 1991; Bagshawe, Br J Cancer 60:275-81, 1989; Bagshawe, et al., Br J Cancer 58:700-3, 1988; Senter, et al., Bioconjugate Chem 4:3-9, 1993; Battelli, et al., Cancer Immunol Immunother 35:421-5, 1992; Pietersz and McKenzie, Immunolog Reviews 129:57-80, 1992; and Roffler, et al., Biochem Pharmacol 42:2062-5, 1991). These techniques can be used for a variety of other specific cell types.

5. Foodstuffs

Also disclosed herein are foodstuffs comprising any of the microcapsules and emulsions disclosed herein. By “foodstuff” is meant any article that can be consumed (e.g., eaten, drank, or ingested) by a subject. In one aspect, the microcapsules can be used as nutritional supplements to a foodstuff. For example, the microcapsules and emulsions can be loaded with vitamins, omega-3 fatty acids, and other compounds that provide health benefits. In one aspect, the foodstuff is a baked good, a pasta, a meat product, a frozen dairy product, a milk product, a cheese product, an egg product, a condiment, a soup mix, a snack food, a nut product, a plant protein product, a hard candy, a soft candy, a poultry product, a processed fruit juice, a granulated sugar (e.g., white or brown), a sauce, a gravy, a syrup, a nutritional bar, a beverage, a dry beverage powder, a jam or jelly, a fish product, or pet companion food. In another aspect, the foodstuff is bread, tortillas, cereal, sausage, chicken, ice cream, yogurt, milk, salad dressing, rice bran, fruit juice, a dry beverage powder, rolls, cookies, crackers, fruit pies, or cakes.

6. Dosage

When used in the above described methods or other treatments, or in the nutritional supplements, pharmaceutical formulations, delivery devices, or foodstuffs disclosed herein, an “effective amount” of one of the disclosed compounds can be employed in pure form or, where such forms exist, in pharmaceutically acceptable salt form and with or without a pharmaceutically acceptable excipient, carrier, or other additive.

The specific effective dose level for any particular subject will depend upon a variety of factors including the disorder being treated and the severity of the disorder; activity of the specific compound employed; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of the compound at levels lower than those required to achieve the desired therapeutic effect and to gradually increase the dosage until the desired effect is achieved. If desired, the effective daily dose can be divided into multiple doses for purposes of administration. Consequently, single dose compositions may contain such amounts or submultiples thereof to make up the daily dose.

The dosage can be adjusted by the individual physician or the subject in the event of any counterindications. Dosage can vary, and can be administered in one or more dose administrations daily, for one or several days. Guidance can be found in the literature for appropriate dosages for given classes of pharmaceutical products. A typical daily dosage of the compounds disclosed herein used alone might range from about 1 to up to about 10 grams or more per day of both algae and cyanobacteria or fractions, extracts, lysates, or derivatives thereof, depending on the factors mentioned above.

(1) Administration and Delivery

In one aspect, disclosed herein are uses of a delivery device to deliver a the compositions disclosed herein to a subject. Further, disclosed are methods for delivering a composition comprising one or more algae species and a cyanobacterium, or fractions, extracts, lysates, or derivatives thereof, to a subject by administering to the subject any of the nutritional supplements, pharmaceutical formulations, delivery devices, and/or foodstuffs disclosed herein.

The compositions disclosed herein (including nutritional supplements, microcapsules, delivery devices, and pharmaceutical formulations) can be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization. Administration of the compositions by inhalant can be through the nose or mouth via delivery by a spraying or droplet mechanism. Delivery can also be directly to any area of the respiratory system (e.g., lungs) via intubation.

### C. METHODS

Disclosed are methods of treating or preventing a viral infection in a subject, comprising administering to the subject the compositions disclosed herein. The viral infection can be treated or prevented by reducing viral load or increasing CD4 levels, for example. Also disclosed is a method of treating or preventing a viral infection in a subject, comprising the steps of: identifying a subject with, or at risk of contracting, a viral infection; and administering to the subject the compositions disclosed herein.

Decreased severity of the viral infection can result in an increased longevity in the subject as compared to a control. For example, the individual can be expected to live 1, 2, 3, 4, 5, 6, 7, 8, 9, 10, or 11 months longer, or 1, 2, 3, 4, 5, 6, 7, 8, 9, 10, 11, 12, 13, 14, 15, 16, 17, 18, 19, 20, 21, 22, 23, 24, 25, or more years longer compared to a control. The decreased severity can also comprise a longer asymptomatic period in the subject as compared to a control. For example, the subject can remain asymptomatic for 1, 2, 3, 4, 5, 6, 7, 8, 9, 10, or 11 months longer, or 1, 2, 3, 4, 5, 6, 7, 8, 9, 10, 11, 12, 13, 14, 15, 16, 17, 18, 19, 20, 21, 22, 23, 24, 25, or more years longer compared to a control. Further, the decreased severity can result in reduced symptoms of the viral infection (e.g., reduced fever, reduced inflammation, and reduced secondary infections.)

The decreased severity can be manifest in a number of different ways. For example, the decreased severity can comprise high CD4 counts as compared to a control. The CD4 count has been used as a measurement to determine the strength of the immune system. It can also be used to judge how far a viral infection is advanced (the stage of the disease), and helps predict the risk of complications and opportunistic infections. The CD4 count can be compared with a count obtained from an earlier test in the same subject. The CD4 count can also be used in combination with the viral load test, which measures the level of HIV in the blood, to determine the staging and outlook of the disease. A CD4 count and a viral load test are usually ordered when a subject is diagnosed with a virus, such as HIV, as part of a baseline measurement. Both tests are commonly repeated about four weeks after starting anti-HIV therapy. If treatment is maintained, a CD4 count can be performed every three to four months thereafter, for example.

Normal CD4 counts in adults range from 500 to 1,500 cells per cubic millimeter of blood. In general, the CD4 count goes down as the viral disease progresses. According to public health guidelines, preventive therapy should be started when an HIV-positive person who has no symptoms registers a CD4 count under 350. The Centers for Disease Control and Prevention considers HIV-infected persons who have CD4 counts below 200 to have AIDS, regardless of whether they are symptomatic.

The decreased severity can also comprise lower HIV viremia levels as compared to a control. Quantitative measurements of HIV viremia in peripheral blood have shown that higher virus levels can be correlated with increased risk of clinical progression of HIV disease, and that reductions in plasma virus levels can be associated with decreased risk of clinical progression. Virus levels in the peripheral blood can be quantitated by direct measurement of viral RNA in plasma using nucleic acid amplification technologies, such as the polymerase chain reaction assay, branched DNA assay and nucleic acid sequence-based amplification assay. These assays quantify human immunodeficiency virus (HIV) RNA levels. Plasma viral load (PVL) testing has become a cornerstone of HIV disease management. Initiation of antiretroviral drug therapy is usually recommended when the PVL is 10,000 to 30,000 copies per mL or when CD4+ T-lymphocyte counts are less than 350 to 500 per mm3 (0.35 to 0.50 3 109 per L). PVL levels usually show a 1- to 2-log reduction within four to six weeks after therapy is started. The goal is no detectable virus in 16 to 24 weeks. Periodic monitoring of PVL is important to promptly identify treatment failure. The same assay can be used for serial PVL testing in the subject. At least two PVL measurements are usually performed before antiretroviral drug therapy is initiated or changed.

Examples of viral infections include, but are not limited to, Herpes simplex virus type-1, Herpes simplex virus type-2, Cytomegalovirus, Epstein-Barr virus, Varicella-zoster virus, Human herpesvirus 6, Human herpesvirus 7, Human herpesvirus 8, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency cirus, Human T-cell Leukemia virus type-1, Hantavirus, Rubella virus, Simian Immunodeficiency virus, Human Immunodeficiency virus type-1, and Human Immunodeficiency virus type-2.

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### D. EXAMPLES

### 1. Example 1

### In Vitro Evidence

Seaweeds and Spirulina extracts in vitro inhibit a variety of enveloped viruses (including herpes simplex virus (HSV)-1 and 2, human cytomegalovirus, measles virus, mumps virus, influenza A virus and human immunodeficiency virus-1 (3, 4, 5). In studies of HIV inhibition, algal extracts disrupt fusion of the V3 loop of gp 120 and can also disrupt transmembrane gp41 (Witvrouw, 1997; Schaeffer, 1999; Luscher-Mattli, 2000; Ayehunie, 1998; Beutler, 2002).

A) Spirulina: In Vitro and In Vivo

Low concentrations (40 μg/mL) of aqueous Spirulina extract (calcium spirulan) in vitro almost completely inhibited HIV-1 adsorption and penetration (Hayashi, 1996). HIV-induced syncytium formation was inhibited at 25 μg/mL. Five times the amount of HIV was required to infect cells that had been pretreated with Spirulina extract. Crude hot water extract concentration of only 0.3 to 1.2 μg/ml reduced HIV-1 replication by 50% (Ayehunie, 1998). No toxicity to uninfected cells was noted.

B) Seaweeds: (Sargassum and Undaria) In Vitro

The most commonly eaten seaweed in Asia, Undaria (“wakame”), and the lesser-known Sargassum are among those that have been tested for anti-HIV activity. A water extract of Undaria (50-1,000 μg/ml) added to HIV infected MT-4 cells for three days resulted in the disappearance of almost all HIV infected cells (Muto, 1992). The same dose strongly inhibited HIV reverse transcriptase activity.) Viva Natural, a commercial water extract of Undaria, suppressed replication of Rauscher virus in BALB/3T3 cells and inhibited syncytia formation. A hot water Sargassum extract inhibited HIV, HCMV, and HSV-1 when added either concurrently or after viral infection (Hoshino, 1998).

### 2. Example 2

### In Vivo Evidence

Initial results from a clinical trial show a reduction in viral load, and an increase in CD4 count in an HIV patient after three weeks daily ingestion of the blue-green alga Spirulina (Arthropira maxima) and brown seaweed (Undaria pinnatifida, called wakame or mekabu in Japanese) capsules.

In a study of Rauscher murine retrovirus-induced erythroleukemia, the water extract of Undaria (Viva Natural) was as effective as AZT in treating the virus. Undaria, unlike AZT, was also effective in preventing infection when given three days before virus inoculation (Furusawa, 1991).

Algal polyanions and mammalian heparin sulfates are similar in structure, although fucoidan, the sulfated polysaccharide and the primary algal polyanion in brown seaweeds, has lower anticoagulant effect than heparin (Matou, 2002). In answer to the question of whether dietary fucoidan from seaweed could be absorbed and physiologically active, Hiebert recently reported that oral heparins could be absorbed in rats (13). Although plasma concentrations were less than 1%, there were physiological changes, as indicated by reduction of thrombosis in the rat jugular vein model. Widespread endothelial cell uptake of oral heparins in addition to circulating plasma concentrations explain this effect. Fucoidans can be similarly absorbed and distributed in the body.

A retrospective dietary assessment of AIDS patients to quantify previous and current algae intake is performed. Such studies are in Japan and Korea, where seaweed is commonly eaten, as well as among the Kanembu tribe of Chad. A prospective trial of Spirulina, Undaria, and/or Sargassum supplementation is conducted among patients for whom the side effects of HAART therapy have resulted in stopping drug therapy, among those who have developed drug resistance, and among those who have either been unable to afford HAART therapy or those who could not adhere to HAART medication schedules. In addition, alga is given with HAART to investigate any enhancement of HAART. Since Spirulina, Undaria, and Sargassum are used as foods, their addition to the diets of people with marginal nutrition can be beneficial based on their protein, vitamin, and mineral content. Addition of either or both of these foods can reduce morbidity and mortality due to AIDS.

### 3. Example 3

### In Vivo Testing

a) Methods

After obtaining Informed Consent, each patient is randomized to one of three treatment regimes. Each subject is given 10 (350-500 mg) capsules or tablets to be taken in the evening with dinner. One group receives 3-5 g/day of Undaria, one group receives 4.25 g/day of Spirulina, and one group receives 1.75 g/day of Undaria and 2-½ g/d of Spirulina. Patients come into the clinic once a week for 3 weeks to have blood drawn, provide a urine specimen, and receive the next week's pills. The MOS-HIV quality of life questionnaire is given at the first and last clinic visit.

Eligible patients are invited to participate in the three-week randomized clinical trial. 21 people with HIV who have either never received anti-HIV therapy by their own choice or because their clinical findings have not yet required it, or have not taken HAART in the last 90 days are enrolled. Eligibility criteria include: serum RNA-HIV level over 10,000 copies/ml, CD4 counts of greater than 350 cells/μL, (unless the patient has refused drug therapy in spite of having CD4 counts of 300 or less), asymptomatic for other infections and diseases, not pregnant, between the ages of 18 and 65 with a weight of at least 110 pounds, not allergic to iodine or seafood, not have symptomatic opportunistic infections or other diseases, and be in stable health.

Blood collection is in 3 pediatric tubes (3 ml). One is in sodium heparin tube and is placed on ice and taken a flow cytometry lab immediately for determination of immunological parameters and lymphocyte activation studies. One is in a silicone-coated tube and sent for viral load determination using a Roche Amplicor HIV-1 monitor. One is in EDTA 7.5% solution tube and is centrifuged at 2500 rpm for 15 minutes, then plasma is aliquoted into cryotubes and frozen at −80.

Patients are enrolled until 21 patients who have completed the entire 3-week study with four blood samples and satisfactory indication of adherence (pill counts and algal pigments detected in urine by fluorometry).

Immunophenotyping: Lymphocyte subsets are enumerated using flow cytometry. Blood is labeled with monoclonal antibodies to lymphocyte subsets CD3, CD4, CD8, CD19, CD56, TCR-gamma delta, CD2, CD38, CD69, CD62P, and CD45 (Beckman-Coulter, Miami, Fla.). Following a 30-minute incubation period, red blood cells are lysed and the specimen acquired on a Beckman-Coulter Excel™ flow cytometer. HIV copies/mL are obtained using the Roche Amplicor HIV-1.

Spot urine samples are collected to investigate the possibility that fluorospectometry can confirm dietary adherence to the algae regiment. This method is adapted from standard environmental algae testing to use with urine.

Randomization is done using random permuted 3 blocks with approximately equal subject characteristics. In the clinic, actual randomization takes place using by the sealed envelope method. Each group has 7 subjects.

Polis et al, (2001) reported that among 124 HIV infected patients who had not yet been treated with HAART, none had baseline viral load counts that varied more than 0.3 log over two baseline measurements. They proposed that if there was not at least a 0.72 log reduction in plasma HIV by the end of day 6, then there was less than 1% chance that a longer course of therapy would work. At the end of a 12-week treatment period, a decrease of 1.5 log or undetectable HIV was classified as a good response to long-term therapy. Paddam (2002) has argued that 6 days was too short, and that four weeks was a better time frame to predict long-term response. Both Polis and Paddam agree that failure to respond to HAART can be seen in the first few weeks. In this study, change in viral load is measured at the end of three weeks. A decrease in viral load of 0.7 log or greater is considered a positive response. To test for a therapeutic effectiveness of 35% with α=0.05 each arm of the study needs 7 subjects. That is, if a positive response for one subject/arm is found in three weeks, the treatment is at least 35% effective.

Simple descriptive statistics are computed to investigate distributions of outcome variables (HIV-1 RNA copies/mL, CD4 counts, and other lymphocyte subpopulations) and to assure that appropriate statistical methods are used (e.g., non-parametric, parametric, transformations if needed to meet model assumptions). A variety of bivariate relationships are investigated between patient characteristics and treatment effects. Mixed models are used to assess multivariate relationships among patient characteristics, treatment, and HIV-1 RNA and CD4 counts. All analyses are conducted using The SAS System (Cary, N.C.).

Undaria pinnatifida is used as the brown seaweed that has been ground and encapsulated into 500 mg gelatin capsules. This seaweed is commonly eaten by people and is available in retail health food stores. Undaria has been tested for iodine content and has 42.5 μg/g. The dose in this study is 5 grams/day, providing 212.5 μg/day additional iodine. The maximum tolerated dose is 1000 μg/day.

Marine Resources harvested the seaweed from the Mercury passage, East coast of Tasmania. It was hand harvested, and then transported to the shore in rope bags, hung on racks in a covered processing facility, then dried using a low heat system at the Marine Resources Pty Ltd facility in Triabunna, Tasmania. It was ground and encapsulated in Sydney, Australia, following all GMP practices. It was shipped to the US by express courier service.

Spirulina tablets have been supplied by Earthrise Farms, in Calipatria, Calif. The Spirulina has been dried within 15 minutes of harvest, and cold-pressed into 500 mg tablets. Earthrise meets or exceeds all national and international food standards in manufacturing quality.

b) Results

Clinical studies showed a 40% reduction in viral load, and 25% increase in CD4 count in an HIV patient after three weeks daily ingestion of the blue-green alga Spirulina (Arthropira maxima) and brown seaweed (Undaria pinnatifida, called wakame or mekabu in Japanese) capsules.

A 60-year-old woman with HIV was recruited to the clinical study of the effects of dietary algae on patients who were HIV positive but not yet qualifying for HAART treatment (viral load over 10,000 and CD4 counts above 350). After an initial clinical evaluation, and obtaining informed consent, she began consuming 5 capsules of the brown seaweed Undaria pinnatifida (623 mg each; 3115 mg) and 5 capsules of Spirulina (Arthrospira platensis) (500 mg each; 2500 mg) each day for a period of three weeks. The subject had co-morbidity of chronic hepatitis C, diabetes and hypertension, as well as HIV (diagnosed in March, 2003). In addition, she was addicted to heroin. She was clinically evaluated once a week for three weeks. No adverse effects were noted, and she reported improved feelings of well-being, increased appetite, healing of a cracked dry skin condition. Her viral counts decreased each week, and after 3 weeks decreased by 40% (HIV RNA QNT BY PCR). Her CD4 counts increased 25%, and CD8 counts increased 23%. Total T cells (CD3) increased 26%. See Table 1 below.

TABLE 1

| | | CD4 | CD8 | CD3 |
|---|---|---|---|---|
| | Viral load | cells/microlitre | cells/microlitre | cells/microlitre |
| Baseline | 72,700 | 446 | 841 | 1301 |
| Week 1 | 65,700 | 559 | 774 | 1356 |
| Week 2* | 58,100 | 339 | 662 | 1034 |
| Week 3 | 43,700 | 559 | 1033 | 1640 |

Undaria pinnatifida is the most commonly eaten seaweed in Japan. The capsules of Undaria pinnatifida used in this study are from Tasmanian mature plants (mekabu). Marinova, the company licensed to harvest Undaria from Tasmanian coastal waters, provides a consistent product of 10% minimum fucoidan. The fucoidan content of this batch was 10.76%. Spirulina was provided by Earthrise Nutritionals, is known to be microcystin (a liver toxin) free, and is grown under fully controlled ponds in southern California.

Active agents Fucoidans are fucose-rich sulfated polysaccharides found in brown seaweeds. They are potent inhibitors of viral entry to cells. Spirulina has likewise shown efficacy in cell culture against HIV, and is similar to seaweed in containing a polyanionic sulfated polysaccharide that is immunostimulatory. However, rhamnose is the major (35%) sugar moiety attached to the sulfated backbone structure. In vitro work on seaweed extracts and Spirulina extracts has demonstrated both inhibition of fusion of HIV to lymphocytes, inhibition of synctium formation, and synergistic activity with AZT, as well as immunostimulatory activity. A fucoidan extract from the Undaria in this study was assessed under the NIAID and found to have good anti-HIV activity. Smaller, organic soluble, or mineral elements of algae can also be used. In vivo work has demonstrated that seaweed (or fucoidan extract) is effective either orally or injected subcutaneously in models of anti-carcinogenesis an effect which may be effected, in part, by increases in chemokines and immune cell populations.

Many polyanions have been tried and shown to be effective against HIV. Algae are relatively low cost, non-drug, food items. The large polysaccharides that are present in algae are thought to be non absorbable. This may not be true for immuno compromised people with “leaky gut.” The gut has a large lymphatic system (gut-associated lymphoid tissues, GALT) which contains over half the body's T cells, and acts as a reservoir for HIV. Uptake of algal components by GALT can inhibit HIV in situ, and perhaps modulate immunity.

Throughout this application, various publications are referenced. The disclosures of these publications in their entireties are hereby incorporated by reference into this application in order to more fully describe the state of the art to which this invention pertains.

### E. REFERENCES

1. UNAIDS 2002. The Report on the Global HIV/AIDS Epidemic (The Barcelona Report) AIDS 2002 XIV International Conference, Barcelona, Jul. 7-12, 2002. http://www.unaids.org/barcelona
2. Abdulqader G, Barsanti L, Tredici M R. Harvest of Arthrospira platensis from Lake Kossorom (Chad) and its household usage among the Kanembu. J Appl Phycology 2000 October; 12 (3-5):493-498, 2000.
3. Hayashi, K, Hayashi T Kojima I. A natural sulfated polysaccharide, calcium spirulan, isolated from Spirulina platensis: In Vitro and ex Vivo evaluation of anti-herpes simplex virus and anti-human immunodeficiency virus activities. AIDS Research and Human Retroviruses 1996 12(15): 1463-71
4. Witvrouw M De Clercq E. Sulfated polysaccharides extracted from sea algae as potential antiviral drugs. Gen Pharmacol. 1997 October; 29 (4):497-511.
5. Schaeffer D J, Krylov V S. Anti-HIV activity of extracts and compounds from algae and cyanobacteria. Ecotoxicol Environ Saf. 2000 March; 45 (3):208-227, doi 10.1006/cesa.1999.1862.
6. Luscher-Mattli M Polyanions—a lost chance in the fight against HIV and other virus diseases Antivir Chem. Chemother. 2000 July; 11(4):249-59
7. Ayehunie S, Belay A, Baba T, Ruprecht R M. Inhibition of HIV-1 replication by an aqueous extract of Spirulina platensis (Arthrospira platensis). Journal of Acquired Immuno Deficiency Syndromes and Human Retrovirology 1998 May 1; 18(1)::7-12
8. Beutler J A, McMahon J B, Johnson T R, O'Keefe B R, Buzzell R A, Robbins D, et. al. High Throughput Screening for Cyanovirin-N Mimetics Binding to HIV-1 gp41. J Biomol Screen. 2002 April; 7 (2):105-10.
9. Muto S, Niimura K, Oohara M, Oguchi Y, Matsunaga K, Hirose K, et. al. Kureha Kagaku Kogyo Kabushiki Kaisha (Tokyo, JP), assignee. Polysaccharides and antiviral drugs containing the same as active ingredient. U.S. Pat. No. 5,089,481. 1992 Feb. 18
10. Hoshino T, Hayashi T, Hayashi K, Hamada J, Lee J B, Sankawa U. An antivirally active sulfated polysaccharide from Sargassum horneri (TURNER) C. AGARDH. Biol Pharm Bull. 1998 July; 21 (7):730-4.
11. Furusawa E, Furusawa S, Chou S C. Antileukemic activity of Viva-Natural, a dietary seaweed extract, on Rauscher murine leukemia in comparison with anti-HIV agents, azidothymidine, dextran sulfate and pentosan polysulfate. Cancer Lett 1991; 56: 197-205
12. Matou S, Helley D, Chabut D, Bros A, Fischer A. Effect of fucoidan on fibroblast growth factor-2-induced angiogenesis in vitro. Thromb Res. 2002 May 15; 106(4-5):213.
13. Hiebert L M. Oral heparins. Clin Lab. 2002; 48(3-4):111-6.
14. Abrams D I, Kuno S, Wong R, Jeffords K, Nash M, Molaghan J B, et. al. Oral dextran sulfate (UA001) in the treatment of the acquired immunodeficiency syndrome (AIDS) and AIDS-related complex. Ann Intern Med. 1989 Feb. 1; 110 (3):183-188.
15. Spieler R. Seaweed compound's anti-HIV efficacy will be tested in southern Africa. Lancet. 2002 May 11; 359(9318):1675.
16. Hirahashi T, Matsumoto M, Hazeki K, Sacki Y, Ui M, Seya T. Activation of the human innate immune system by Spirulina: augmentation of interferon production and NK cytoxicity by oral administration of hot water extract of Spirulina platensis. Int Immunopharmacol 2002 March; 2(4):423-434.
17. Beljaars L, Floris R, Berkhout B, Smit C, Meijer D K, Molema G. The influence of charge clustering on the anti-HIV-1 activity and in vivo distribution of negatively charged albumins. Biochem Pharmacol. 2002 May 1; 63(9):1663-73.
18. Shan B E, Yoshida Y, Kuroda E, Yamashita U. Immunomodulating activity of seaweed extract on human lymphocytes in vitro. Int J Immunopharmacol 1999 January; 21(1):59-70
19. Weiner M L: Intestinal Transport of some macromolecules in food. Food Chem Toxi 1988, October; 26(10):867-80.
20. Sciammas R, Kodukula P, Tang Q, Hendricks R L, Bluestone J A, 1997. T cell receptor-gamma/delta cells protect mice from herpes simplex virus type 1-induced lethal encephalitis J Exp Med: 185(11):1969-1975.
21. Anastase-Ravion S, Carreno M P, Blondin C, et al. Heparin-like polymers modulate proinflammatory cytokine production by lipopolysaccharide-stimulated human monocytes. J Biomed Mater Res. 2002 Jun. 5; 60(3):375-83.
22. Arasaki S and Arasaki T. Vegetables from the Sea. Japanese Publications, Tokyo. 1983. p. 133.
23. Ayehunie S, Belay A, Baba T, Ruprecht R M. Inhibition of HIV-1 replication by an aqueous extract of Spirulina platensis (Arthrospira platensis). Journal of Acquired Immuno Deficiency Syndromes and Human Retrovirology 1998 May 1; 18(1):7-12
24. Belay, A. The potential application of Spirulina (Arthropira) as a nutritional and therapeutic supplement in Health management. J Am Nutraceut Assoc. 2002; Spring 5(2): 27-48.
25. Beutler M, Wiltshire K H, Meyer B, et. al. A fluorometric method for the differentiation of algal populations in vivo and in situ. Photosynthesis Research 2002 72(1): 39-53
26. Cooper, R, Dragar C, Elliot K, et. al. GFS, a preparation of Tasmanian Undaria pinnatifida is associated with healing and inhibition of reactivation of Herpes. BMC Complement Altem Med. 2002 Nov. 20; 2(1):11.
27. Delpeuch, F, Joseph A, Cavelier C. Consommation alimentaire et apport nutritionnel des algues bleues (Oscillatoria platensis) chez quelques populations du Kanem (Tchad). Ann Nutr Alim; 1976, 29, 497-546.
28. Furusawa E, Furusawa S, Chou S C. Antileukemic activity of Viva-Natural, a dietary seaweed extract, on Rauscher murine leukemia in comparison with anti-HIV agents, azidothymidine, dextran sulfate and pentosan polysulfate. Cancer Lett 1991; 56: 197-205
29. Hirahashi T, Matsumoto M, Hazeki K, et. al. Activation of the human innate immune system by Spirulina: augmentation of interferon production and NK cytoxicity by oral administration of hot water extract of Spirulina platensis. Int Immunopharmacol. 2002 March; 2(4): 423-434.
30. Hoshino T, Hayashi T, Hayashi K, et al. An antivirally active sulfated polysaccharide from Sargassum horneri (TURNER) C. AGARDH. Biol Pharm Bull. 1998 July; 21 (7): 730-4.
31. Luscher-Mattli M Polyanions—a lost chance in the fight against HIV and other virus diseases? Antivir Chem Chemother. 2000 July; 11(4):249-59
32. Okai Y, Ishizaka S, Higashi-Okai K, Yamashita U. Detection of immunomodulating activities in an extract of Japanese edible seaweed, Laminaria japonica (Makonbu). J Sci Food Agric 1996, 72: 455-460.
33. Saker K E, Allen V G, Fontenot J P, et al. Tasco-Forage: II. Monocyte immune cell response and performance of beef steers grazing tall fescue treated with a seaweed extract. J Anim Sci. 2001 April; 79(4): 1022-31.
34. Witvrouw M De Clercq E. Sulfated polysaccharides extracted from sea algae as potential antiviral drugs. Gen Pharmacol. 1997 October; 29 (4): 497-511.
35. Ayehunie S, Belay A, Baba T W, Ruprecht R M. Inhibition of HIV-1 replication by an aqueous extract of Spirulina platensis (Arthrospira platensis). J Acquir Immune Defic Syndr Hum Retrovirol 1998; 18(1):7-12.
36. Delpeuch F, Joseph A, Cavelier C. Consommation alimentaire et apport nutritonnel des algues bleues (Oscillatoria platensis) chez quelques populations du Kanem (Tchad). Ann Nutr Aliment 1976; 29:497-516.
37. Schaeffer D J, Krylov V S. Anti-HIV activity of extracts and compounds from algae and cyanobacteria. Ecotoxicol Environ Saf. 2000; 45(3):208-27.
38. Yamamoto I, Maruyama H, Takahashi M, Komiyama K. The effect of dietary or intraperitoneally injected seaweed preparations on the growth of sarcoma-180 cells subcutaneously implanted into mice. Cancer Lett. 1986; 30(2):125-31.

1. A composition comprising cyanobacteria and one or more types of algae.

2. A dietary supplement comprising cyanobacteria and one or more types of algae.

3. The composition of claim 1, wherein the composition also comprises a pharmaceutically acceptable carrier.

4. A composition comprising fractions of cyanobacteria and one or more types of algae.

5. A composition comprising extracts of cyanobacteria and one or more types of algae.

6. A composition comprising lysates of cyanobacteria and one or more types of algae.

7. A composition comprising derivatives of cyanobacteria and one or more types of algae.

8. The composition of claim 4, wherein the fractions are active fractions.

9. The composition of claim 5, wherein the extracts are active extracts.

10. The composition of claim 6, wherein the lysates are active lysates.

11. The composition of claim 7, wherein the derivatives are active derivatives.

12. The composition of claim 5, wherein the extract of the algae comprises algal polyanions.

13. The composition of claim 1, wherein the algae is selected from the group consisting of undaria, alaria, and sargassum.

14. The composition of claim 1, wherein the cyanobacteria is spirulina.

15. A method of treating or preventing a viral infection in a subject, comprising administering to the subject the composition of claim 1.

16. The method of claim 15, wherein viral infection is treated or prevented by reducing viral load.

17. A method of increasing CD4 levels in a subject comprising administering to the subject the composition of claim 1.

18. A method of treating or preventing a viral infection in a subject, comprising the steps of:
a. identifying a subject with, or at risk of contracting, a viral infection; and
b. administering to the subject the composition of claim 1.

19. The method of claim 15, wherein the viral infection is a retrovirus.

20. The method of claim 19, wherein the retrovirus is HIV.
